(19) United States
(12) Patent Application Publication
(10) Pub. No.: US 2002/0142992 A1
(43) Pub. Date: Oct. 3, 2002

(54) Cellulosic foam compositions

(76) Inventor: George H. Scherr, Park Forest, IL (US)

Correspondence Address: George H. Scherr, Ph.D., 33 Monee Road, Park Forest, IL, 60466, US

(21) Appl. No.: 09/818,928

(22) Filed: Mar. 28, 2001

(51) Int. Cl. A61K 31/732; C08B 37/06

(52) U.S. Cl. 514/54; 536/2

(57) ABSTRACT

The invention described herein relates to the preparation of cellulosic foam products prepared from pectin and pectin derivatives and the process for preparing them. The cellulosic foam products are amenable for use in the preparation of medical and veterinary dressings for the treatment of wounds and related pathological states.

### DESCRIPTION OF THE PRIOR ART

U.S. Pat. No. 4,347,261 by Challen, et al. describes the use of edible alginate and low-methoxy-pectate gels which are produced by mixing aqueous slurries containing finely divided foodstuff and particles of calcium sulphate, which slurry now has the capability of slowly releasing gelling agents into solution. The mixing of the sodium alginate and/or pectins into the aqueous slurry of the finely divided foodstuff has to take place before the release of the calcium ions in solution; the total addition of the calcium sulphate being required before gellation begins. There is no foam produced as a result of the gellation described in U.S. Pat No. 4,347,261, nor in fact is there one desired. The inventors therein also stipulate that the ratio of the volume of water in the sol to the water available in the slurry would have to be greater than 1.5:1.

In the U.S. Pat No. 4,347,261 the inventors claim that the rapid mixing of particles of solid calcium sulphate uniformly throughout an aqueous alginate or low-methoxy pectate sol and then allowing the mixture to gel under shear-free conditions can produce an excellent gel in a short time.

U.S. Pat. No 5,688,923 claims to be the first development for the commercial process of making pectin fibers that might be suitable for use in wound dressing applications (U.S. Pat. No. 5,688,923; column 1, lines 8-12). The pectins utilized in U.S. Pat. No. 5,688,923 were calcium-ion-sensitive in order to achieve the feasibility of using a spinneret through which the solution of the pectin is forced into a bath containing calcium or other polyvalent cations that would precipitate the pectin in the form of a filament, by what is commonly known in the profession as the “wet process.” The authors of U.S. Pat. No. 5,688,923 indicate (Column 1, paragraph 6) that, prior to their invention, there existed in the industry a need for a simple and reproducible process for making pectin fibers that have properties suitable for use in wound dressing applications and that such properties would include “high tensile strength, softness, stability in a wound environment, non-brittleness, sterilizability, a high level of wet strength and resilience.” The authors purport to have achieved these properties by the following:

(a) Preparing a solution of the pectin to be utilized in the spinning process and filtering through a 5 μ filter in combination with centrifugation in order to remove undissolved particles that might clog the spinneret.

(b) Following filtration, the solution of calcium-sensitive pectin is pumped at a suitable pressure through a spinneret into a spinning solution containing calcium chloride.

(c) The calcium chloride concentration in the precipitating bath has to be at a concentration resulting in a viscosity of the calcium chloride that is significantly greater than the density of the pectin precipitate or at a concentration resulting in a density of the calcium chloride solution significantly lower than the density of the pectin precipitate, depending upon whether one desires the calcium pectin fibers to sink to the bottom of the tank or be drawn up towards the top of the tank.

(d) The spinning of the pectin compounds takes place in an aqueous bath containing the calcium salt and in a preferred embodiment, isopropyl alcohol is added to the aqueous solution of calcium chloride in order to maintain a relatively low density of the spinning solution so that the fibers would sink to the bottom of the calcium chloride bath.

(e) Once the pectin fibers have been precipitated as the calcium salt of the pectin compounds, the fibers have to be washed with water or a water-alcohol mixture to remove excess calcium chloride, and the fiber may then be wet-drawn to improve its tensile strength and to reduce its denier.

(f) Fibers are usually dried overnight in a vacuum following which they may be utilized to prepare either woven or non-woven dressings.

(g) If the pectin fibers are to be used to prepare woven dressings, they would have to be prepared in the form of threads which are then woven on a suitable machine. If the pectin derivative fibers are to be utilized in non-woven dressings, they are usually cut to the desired staple size, and then carded in a carding machine prior to their being formed into a non-woven dressing.

The present invention obviates all of the steps above and still makes possible the fabrication of a calcium precipitated pectin dressing that contains the properties of high tensile strength, softness, stability in a wound environment, non-brittleness, sterilizability, a high level of wet strength, and resilience.

### SUMMARY OF THE INVENTION

The present invention relates to a foam preparation which comprises a calcium-sensitive pectin moiety in which the calcium ion is contained in a calcium salt of relatively high solubility. The invention described herein also comprises a process in which:

(a) A composition of the calcium sensitive pectin is dissolved in water utilizing heating if necessary, and;

(b) Adding to the pectin solution, a suitable quantity of ammonium hydroxide, a calcium salt of a relatively high solubility, and utilizing biologically acceptable reagents which produce a foam, so that;

(c) When a viscous foamed composition is layered onto a plate or onto a suitable backing, and left to dry at room temperature, or in an oven if desirable, a dried sterilizable foam calcium pectin dressing is prepared.

The foam pectin dressing so prepared is soft, has a high tensile strength, has excellent stability in a wound environment, is non-brittle, is sterilizable, has a high level of wet strength, as well as resilience.

### Attributes of the Invention Described Herein

In order to dress wounds on various parts of the human or animal body, it is frequently necessary to utilize a dressing which can be draped around a small circumference such as a finger or an arm. Such a dressing should have excellent resilience and a degree of elasticity so that it does not fracture when subjected to the stress required by dressing areas of the body of relatively small circumference. Consequently, one of the salient advantages discovered in the preparation of the pectin compositions suitable for medical or veterinary dressings as described herein, utilizes the addition of sodium tetraborate to the pectin composition which significantly enhances the elasticity of the dressing so prepared.

The other advantage of the invention described herein relates to the preparation of foam pectin compositions in which the aqueous portion of the composition can be removed by air-drying or regulated heat drying without the necessity of utilizing an expensive freeze-drying apparatus for its preparation

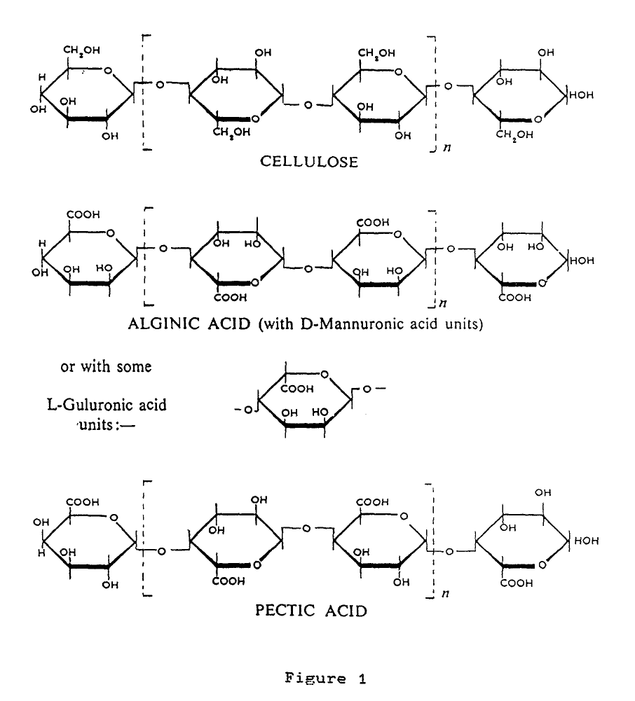

Pectins are cellulosic molecules as are the alginates, both of which are derivatives of their respective molecules in the acid state (See FIG. 1). A salient advantage of the composition described herein stems from the observation that the spacial configuration of the molecular structure of the pectin is sufficiently similar to the spacial configuration of the molecular structure of alginate. Thus a mixture of the two should permit the utilization of a composition in which the intramolecular binding between pectin molecules and alginate molecules is feasible and would be expected to enhance the tensile strength of a product prepared from pectin alone since alginate produced fibers have a significantly higher tensile strength than the equivalent product made solely of pectin fibers.

Another salient advantage of the invention described herein concerns the feasibility of adding ingredients to the pectin composition, which ingredients may contain properties such as being particulate, having high viscosity, or having or resulting in a rheology which makes it undesirable or unfeasible for such compositions to be forced through a fine spinneret to produce the pectin fibers as currently practiced in U.S. Pat. No. 5,688,923.

Pectins can also be prepared with a degree of esterification (DE) which varies depending upon the esterification of the carboxyl groups of the pectin molecule. Since it is desirable in preparing a pectin moiety that it may be amenable to being precipitated with a polyvalent cation that cross-links with the pectin, then the degree of esterification should be minimized so that it should not interfere with the gelling reaction when certain polyvalent cations are added to the pectin. The polyvalent cation for cross-linking the pectins is preferably a calcium ion. The DE of the pectin should be less than 50% so that a lower concentration of calcium and/or other polyvalent cations which precipitate the pectin may be utilized. In practice it has been found that the DE of pectins being less than 20% are the more desirable since lower calcium ion concentrations may be utilized for their gelation thus reducing the cost of manufacture.

Pectins that are esterified can be converted to amidated pectins by reacting them with ammonium hydroxide as is well known in the profession, and such amidated pectins should preferably have a low degree of amidation in order to react with calcium or other divalent cations which can precipitate the pectins as gels. The amidated pectins preferably should have a degree of amidation of approximately 25% for more optimum reaction with calcium ions in forming gels.

Because dressings to be used in the treatment of injuries and lesions, especially those that may be frankly bleeding or exudating serum, would require a secondary dressing to be placed over the pectin foam dressing prior to the dressing being affixed to the body with an adhesive tape, it would therefore be very desirable to have a simple, inexpensive method of affixing a backing to the pectin dressing which can become an integral part of the pectin-precipitated-foam dressing. This would obviate the necessity of having to purchase and sterilize two separate dressings and also stocking two separate dressings.

It is thus another salient advantage of the invention described herein that the pectin composition may be layered onto a woven or non-woven fiber backing which, if containing a sufficiently coarse fiber network will entrap the pectin foam composition when it is dried and thus result in a composite foam dressing attached to a suitable backing which can be placed directly on a wound and appropriately taped into position.

The use of desirable particulate matter such as micro-particles that can act as time-release particles, aqueous insoluble medicaments, or even the use of intact cells such as yeast cells, blood cells, or human or animal tissue cells, that might be desirable to apply to an open wound may be introduced into the pectin foam composition described herein. As is well known in the profession, the pH of the pectin composition may have to be adjusted to be commensurate with the cells that are to be incorporated in the final composition, and such pH adjustment is readily made by those skilled in the art. Such particulate matter and/or aqueous insoluble matter which can be incorporated into the pectin foam composition described in our patent is an attribute not feasible when pectin fibers are prepared by a spinning process.

Having set forth the tenets of the invention contained herein, the following non-limiting examples illustrate various compositions that are inherent in our invention.

### DESCRIPTION OF THE PREFERRED EMBODIMENTS

### EXAMPLE 1

Twenty grams of calcium-sensitive pectin such as pectins which have a degree of esterification of less than 30 per cent which may be procured from Sigma Chemical Company or from Hercules, Inc., is dissolved in 1500 ml of hot deionized water that has been heated to between 80°-90° C. With continuous stirring, add to the solution of pectin, 7.0 grams of calcium sulphate (CaSO₄. 2H₂O) and 75 ml of glycerin.

Following vigorous stirring until all of the ingredients have been dissolved and thoroughly mixed, add

400 ml of deionized water,

7.5 grams of sodium bicarbonate (NaHCO₃)

6.0 ml of the surface active agent polyoxyethylenesorbitan monooleate (Tween 80®, Atlas Chemical Industries, Inc.)

6.0 ml of the surface active agent polyoxyethylene-polyoxypropylene block polymer (L64, (Wyandotte Corp.)

To the above mixture now add slowly by pipetting and with continuous stirring 10 ml of a dilute solution of acetic acid prepared by diluting one part of glacial acetic acid with two parts of deionized water.

Following the addition of the diluted acetic acid, the composition will foam and gradually become more viscous.

The pectin composition thus prepared will contain a considerable amount of foam, which foam which will not rise to the surface of the pectin composition, because the viscosity of the final pectin mixture is greater than the buoyancy of the foam. When poured onto a plate such as one made of plastic or metal, the dish may be air-dried or placed into a drying oven on the following schedule of drying:

70° C.-2 hours

60° C.-2 hours

40° C.-until dry

Alternatively, pectin compositions prepared as above, can be poured onto a backing composed of a non-woven cotton-rayon mixture or a polyester non-woven backing, so that an amount of the alginate foam mixture partially penetrates into the fiber and thus when dry, will retain this backing as part of the dried finished dressing.

It is thus a salient advantage of the patent described herein that the dried pectin foam composition, when ready to be cut into appropriate sized dressings, packaged, and sterilized ready for use, already has a backing affixed to the pectin foam composition and does not require any additional secondary sterile backing after the sterile pectin dressing is placed on a wound.

### EXAMPLE 2

It is frequently desirable to decrease the viscosity of the pectin composition as described in Example 1 in order to more readily facilitate layering the composition onto a tray for drying or onto an appropriate backing so that the more fluid composition can penetrate the fibers of the backing to a degree which will result in an enhanced entrapment of the fibers in the pectin dressing after the composition is appropriately dried. Consequently, the composition as described in Example 1 is prepared and to it, just prior to the addition of the dilute acetic acid, is added 20 ml of ammonium hydroxide which reduces the viscosity of the solution and has the attribute that, during the drying phase of the layered pectin composition, the ammonia evaporates so leaving no residual except for the water contained in the 20 ml of ammonium hydroxide.

### EXAMPLE 3

The pectin composition prepared as described in Example 2 above, with continuous vigorous stirring has added to it 7.5 grams of sodium tetraborate (Na₂B₄O₇. 10H₂O) dissolved in 100 ml of deionized water at a point just prior to the addition of the dilute acetic acid. The mixture is continuously stirred and then may be layered onto dishes for drying or layered onto a suitable backing as described in Example 1 above.

### EXAMPLE 4

The composition described above in Example 1 has added to it following the addition of L64, 60 ml of a 2.5% solution of sodium alginate, (Kelco brand HV), and 10 ml of a 1% solution of ammonium alginate (Kelco brand Kelcoid). When all of the ingredients have been thoroughly dispersed by vigorous mixing, the diluted acetic acid is added as indicated in Example 1 which will then react with the sodium bicarbonate resulting in a foam composition which can then be layered onto trays containing a suitable backing as described in Example 1 above.

### EXAMPLE 5

The zinc salt of bacitracin, having a concentration of 67 IU/mg, is added in an amount of 230 mg to 10 ml of deionized water. Neomycin sulphate powder assaying as 704 mog neomycin/mg of material is added to 10 ml of deionized water in an amount of 135 mg. Polymyxin B sulphate containing 8547 units of polymyxin B/mg of powder is added to 10 ml of deionized water in an amount of 22.6 mg.

The three separate solutions are stirred until all of the antibiotics have been dissolved and then they are mixed, to form a total of 30 ml of solution. The mixture of antibiotics so prepared is now added to the composition described in Example 1 after the addition of the 6 ml of L64.

To the above mixture now add slowly by pipetting and with continuous stirring 10 ml of a dilute solution of acetic acid prepared by diluting one part of glacial acetic acid with two parts of deionized water.

Following the addition of the diluted acetic acid, the composition will gradually become more viscous.

The pectin composition with antibiotics can then be poured onto a plate or onto a suitable backing and dried as set forth in Example 1.

### EXAMPLE 6

Following the addition of the acetic acid as described in Example 1 above, with continued stirring, add 1.5 grams of a highly hydrophillic preparation called “Drimop” manufactured by MultiSorb Technologies which is a sodium polyacrylate polymer.

The sodium polyacrylate polymer is highly hydrophilic and therefore, an amount of water would be retained by this product, even after drying in accord with the process set forth in Example 1. The unique value of adding a small amount of a highly hydrophilic preparation results in the retention of bound water to such a hydrophilic agent as the sodium polyacrylate polymer which thus results in a dressing having an amount of moisture which retains a ‘cool’ touch when the dressing is applied to an open wound. The “Drimop” sodium polyacrylate polymer being highly hydrophilic, also enhances the moisture-absorbing capacity of the dressing when it is applied to an open and/or exudating wound. The rest of the procedure following the addition of Drimop is as described in Example 1 which permits the preparation of the dressing with or without a backing.

### EXAMPLE 7

The components as set forth in Example 1 are followed until that point in the addition of the diluted acetic acid. Following the addition of the diluted acetic acid, and with continued and vigorous stirring, 35 grams of maltodextrin with a dextrose equivalent of 13.0 to 17.0 prepared by Aldrich Chemical Company, Inc. are added. The ingredients are stirred vigorously with a stirrer until the composition becomes viscous and to this composition is added 20 ml of ammonium hydroxide and 150 ml of deionized water. The semi-solid foamed composition may be continuously stirred until it is ready to pour onto the surface of a plate where it can be dried at room temperature or in an oven as described in Example 1. Alternatively, the composition can be layered onto a gauze, cotton, or polyester backing where, when dried, it will adhere to and become affixed to the fibers of the backing. The dried finished dressing can be cut, sealed into suitable packages as is well known in the profession and sterilized and stored in hospital settings to be used when required.

The pectin component in contact with an open wound, especially one which is exudating serum or blood, will gradually become hydrocolloidal, will permit the continuous diffusion of the maltodextrin to the site of the wound, and will retain all of the clinical advantages that are delineated in U.S. Pat. Nos. 5,177,065 and 4,778,679.

### EXAMPLE 8

The ingredients that are described in Example 7 above are prepared in the same way and to the semi-solid foamed composition is added 0.5 grams of ascorbic acid, to provide the beneficial effect of ascorbic acid as it is described in U.S. Pat. Nos. 5,177,065 and 4,778,679.

### EXAMPLE 9

The pectin composition as described in Example 1 above, is prepared with stirring and to the final foamed pectin composition is added a dispersion of 10.0 ml of bovine collagen having a concentration of 100 mg per milliliter of solution.

This composition can now be dried and layered with or without a backing as described in Example 1 above.

### EXAMPLE 10

Following the addition of acetic acid, as set forth in Example 1 above, the pectin composition may then be sterilized in a suitable container by ionizing or E-beam sterilization or in fact by the addition of anti-microbial agents to produce a sterile composition of the pectin.

Using established aseptic techniques, a cell suspension is prepared from a culture of human, animal, or microbial cells and are aseptically harvested into a suitable buffered medium.

The cells thus suspended in a medium are then aseptically added to the sterile pectin foam slurry as prepared above, with slow mixing.

The cell containing pectin foam slurry can then aseptically be layered onto a sterile sheet so that when dried, may be cut into appropriate sizes as required.

The above descriptions and examples illustrate particular constructions including the preferred embodiments of the solutions. However, the invention is not limited to the precise constructions described herein, but, rather, all modifications and improvements thereof encompassed within the scope of the invention.

Many of the examples described herein utilize the surface active agents such as those characterized as Tween 80 or Pluronic L64. These surface-active agents are utilized primarily to effect a dispersion between the non-aqueous miscible components utilized in achieving a coercive mixture with the aqueous soluble pectins in order to insure a homogeneity throughout the solutions that are then precipitated as insoluble pectate compositions.

These surface active agents are also utilized in order to improve the wetting of a medical dressing or bandage in the event that a wound may be exudating, and the enhanced wicking in such a bandage or medical dressing serves to quickly absorb any blood or serum from a wound site into the dressing. Other surface active agents, such as the Na salt of dodecyl SO₄ (sodium lauryl sulfate) or a member of the group of Tweens: (Tween 20, polyoxyethylene sorbitan monolaurate; Tween 40, polyoxyethylene sorbitan monopalmitate; or Tween 85, polyoxethylene sorbitan trioleate may be incorporated into the pectin composition without deviating from the novelty of the invention described herein.

Note that in the examples cited herein, the effervescent compound that reacts with the water soluble dilute acetic acid with the resultant evolution of gases which become entrapped in the formation of the gel network is sodium bicarbonate. Other water soluble effervescent compounds may be utilized and other acids may be utilized to produce the evolution of gases which become entrapped in the pectin gel network without deviating from the novelty of the invention described herein. Thus, various water insoluble metal salts that can react with water soluble acids are calcium carbonate, calcium phosphate dibasic, barium carbonate, or zinc carbonate. Examples of suitable acids would include lactic acid, maleic acid, gluconic acid, ascorbic acids, and hydrochloric acid.

Should it be desirable to utilize gases other than carbon dioxide to form the foam that forms the stable hydrogel composition described herein, inert gases such as nitrogen or argon, or other gases may be directly introduced from compressed gas cylinders into the pectin composition described in the claims herein as long as the pectin compositions described have a viscosity greater than the buoyancy of the gases to be entrapped therein. The addition of such other gases will cause the formation of stable hydrogel pectin foam compositions in accord with the novelty of the invention described herein.

In the examples cited herein, calcium sulphate has been utilized to provide the calcium ions which precipitates the aqueous insoluble calcium pectate which insolubilization may also serve to entrap into the calcium pectate matrix other components as described herein. It is clear, as has been mentioned, that other salts may be utilized to precipitate the pectins such as those of aluminum, zinc, copper, chromium, or silver and these insoluble pectates may be readily utilized to precipitate the various mixtures described in the examples provided herein without deviating from the essential merits of this invention. However, if the pectate compositions are to be utilized in or on biological tissues, the particular salt utilized to precipitate the pectins should be dictated by any restraints of toxicity or other untoward reactions that might result from their use for the preparation of bandages, dressings, or surgical products.

In the examples cited herein, bovine collagen has been utilized to provide a haemostatic agent in the event of frank bleeding of a wound. It is clear, that collagens other than bovine collagen may be utilized for this purpose such as porcine collagen or human collagen without deviating from the essential merits of this invention.

In the examples cited, antibiotics have been utilized to reduce microbial growth in and/or on the wound. It is clear that other antimicrobial substances may suitably be utilized without deviating from the essential merits of this invention. However, the antimicrobial agents used may be dictated by the scope of the injury and/or the type of infection, such as bacterial or fungal that would dictate specific antimicrobial agents for use in stemming the infection.

Such other antimicrobial agents may be utilized without deviating from the essential merits of this invention.

The examples cited a hydrophilic substance such as the sodium polyacrylate polymer designated as “Drimop” may be utilized to retain bound water in the dressing. It is clear that other hydrophilic preparations may be utilized with restrictions only dictated by their safety when incorporated into a dressing which is to be utilized in and on a wound, without in any way deviating from the essential tenets of the invention described herein.

The sodium alginate principally utilized in the examples described herein was one having an aqueous viscosity of 753 cP at 1.25% concentration. It is clear that other sodium alginates having other viscosities may be utilized without deviating from the novelty of the revelations contained in this patent as long as the alginate is of a concentration and viscosity that can be reasonably poured into a mold when a calcium or other anion alginate precipitating molecule is added to the sodium alginate.

Although the alginate used in the examples described herein was sodium alginate, it is clear that other water soluble alginates may be utilized without deviating from the novelty of the invention described herein such as water soluble ammonium alginate, magnesium alginate, or potassium alginate.

In the examples cited, the pectin had a degree of esterification of less than 30% in order to achieve a relatively high degree of reactivity with calcium ions. It is clear however, that pectins other than those having a degree of esterification of less than 30% may be utilized and pectins having a degree of amidation other than 25% may be utilized without deviating from the essential tenets of the invention.

I claim:

1. The process for making a polyvalent cation cross-linked pectin foam composition comprising:
a) dissolving in water either a low esterified calcium sensitive pectin having a degree of esterification (DE) of less than 30% and which pectin has a degree of amidation of less than 50%;
b) with continuous stirring, adding to the dissolved pectin a polyvalent cation; a plasticizing agent, the soluble effervescent compound; and
c) adding a surface active agent and with continuous stirring adding a suitable acid that will liberate a gas from the soluble effervescent compound, which gas will become entrapped in the pectin composition which has been insolubilized with the polyvalent cation.

2. The process of claim 1 wherein non-pectin polysaccharides are added to the pectin composition.

3. The process of claim 2 wherein the polysaccharide is selected from the group consisting of carboxymethylcellulose, carboxymethyl ethyl cellulose, hyaluronic acid, carrageenan, alginic acid, sodium alginate, and gellan gum.

4. The process of claim 1 wherein the polyvalent cation is selected from a metal ion derived from salts selected from the group consisting of alkaline earth metal salts, alkali metal salts, transistion metal salts, and mixtures thereof.

5. The process of claim 4 wherein the polyvalent cation is selected from the group consisting of calcium, barium, magnesium, zinc, iron, aluminum, copper, strontium, manganese, and mixtures thereof.

6. The process of claim 5 wherein the cation is selected from the group consisting of calcium, barium, copper, zinc, iron, aluminum, and mixtures thereof.

7. The process of claim 6 wherein the cation is calcium.

8. The process of claim 1, wherein the upper limit of the DE of the calcium sensitive pectin is 50%.

9. The process of claim 1, wherein the upper limit of the DE of the calcium sensitive pectin is 30%.

10. The process of claim 1, wherein the lower limit of the DE of the calcium sensitive pectin is 0%.

11. The process of claim 1 wherein the lower limit of the DE of the calcium sensitive pectin is 5%.

12. The process of claim 1 wherein the lower limit of the DE of the calcium sensitive pectin is 10%.

13. The process of claim 1 wherein the upper limit of the degree of amidation (DA) of the calcium sensitive pectin is 40%.

14. The process of claim 1 wherein the upper limit of the DA of the calcium sensitive pectin is 25%.

15. The process of claim 1 wherein the upper limit of the DA of the calcium sensitive pectin is 20%.

16. The process of claim 1 wherein the lower limit of the DA of the calcium sensitive pectin is 0%.

17. The process of claim 1 wherein the lower limit of the DA of the calcium sensitive pectin is 5%.

18. The process of claim 1 wherein the lower limit of the DA of the calcium sensitive pectin is 10%.

19. The process of claim 1, wherein the calcium sensitive pectin is derived from citrus pectin.

20. The process of claim 19 wherein the citrus pectin is selected from the group consisting of lime, lemon, grapefruit, and orange.

21. The process for a wound dressing prepared in accord with claim 1 in which a medicament is present in the polyvalent cation cross-linked pectin foam composition.

22. The process for a wound dressing prepared in accord with claim 21 in which the medicament is collagen.

23. The process for a wound dressing prepared in accord with claim 21 in which the medicament is maltodextrin.

24. The process for a wound dressing prepared in accord with claim 21 in which the medicament is an antibiotic.

25. The process for a wound dressing prepared in accord with claim 21 in which the medicament is an antibacterial agent.

26. The process for a wound dressing prepared in accord with claim 21 in which the medicament is a hydrophilic substance.

27. The process for a wound dressing prepared in accord with claim 26 in which the hydrophilic substance is a sodium acrylic polymer.

28. The process for a wound dressing as set forth in claim 1 in which a backing is affixed to the polyvalent cation cross-linked pectin foam composition.

29. The process for a wound dressing of claim 21, in which the backing is an adhesive backing.

30. The process for a wound dressing of claim 21, in which the backing is a non-woven backing.

31. The process for a wound dressing prepared in accord with claim 28 in which a medicament is present in a polyvalent cation cross-linked pectin foam composition comprising a calcium sensitive low methoxy pectin with a degree of esterification (DE) of less than 50% and having a DA of less than 50%.

32. A polyvalent cation cross-linked pectin foam composition comprising a calcium sensitive low methoxy pectin with a degree of esterification (DE) of less than 50%.

33. The composition of claim 32 wherein non-pectin polysaccharides are added to the pectin composition.

34. The composition of claim 33 wherein the polysaccharide is selected from the group consisting of carboxymethylcellulose, carboxymethy ethyl cellulose, hyaluronic acid, carrageenan, alginic acid, sodium alginate, gellan gum, and mixtures thereof.

35. The composition of claim 32 wherein the polyvalent cation is selected from a metal ion derived from salts selected from the group consisting of alkaline earth metal salts, alkali metal salts, transistion metal salts, and mixtures thereof.

36. The composition of claim 35 wherein the polyvalent cation is selected from the group consisting of calcium, barium, magnesium, zinc, iron, aluminum, copper, strontium, manganese, and mixtures thereof.

37. The composition of claim 36, wherein the cation is selected from the group consisting of calcium, barium, copper, zinc, iron, aluminum, and mixtures thereof.

38. The composition of claim 37, wherein the cation is calcium.

39. The composition of claim 32, wherein the upper limit of the DE of the calcium sensitive pectin is 50%.

40. The composition of claim 32, wherein the upper limit of the DE of the calcium sensitive pectin is 30%.

41. The composition of claim 32, wherein the lower limit of the DE of the calcium sensitive pectin is 0%.

42. The composition of claim 32 wherein the lower limit of the DE of the calcium sensitive pectin is 5%.

43. The composition of claim 32 wherein the lower limit of the DE of the calcium sensitive pectin is 10%.

44. The composition of claim 32 wherein the upper limit of the degree of amidation (DA) of the calcium sensitive pectin is 40%.

45. The composition of claim 32 wherein the upper limit of the DA of the calcium sensitive pectin is 25%.

46. The composition of claim 32 wherein the upper limit of the DA of the calcium sensitive pectin is 20%.

47. The composition of claim 32 wherein the lower limit of the DA of the calcium sensitive pectin is 0%.

48. The composition of claim 32 wherein the lower limit of the DA of the calcium sensitive pectin is 5%.

49. The composition of claim 32 wherein the lower limit of the DA of the calcium sensitive pectin is 10%.

50. The composition of claim 32, wherein the calcium sensitive pectin is derived from citrus pectin.

51. The composition of claim 50 wherein the citrus pectin is selected from the group consisting of lime, lemon, grapefruit, and orange.

52. The composition for a wound dressing as set forth in claim 32 in which a backing is affixed to the polyvalent cation cross-linked pectin foam composition.

53. The composition for a wound dressing as set forth in claim 52 wherein non-pectin polysaccharides are added to the pectin composition.

54. The composition for a wound dressing as set forth in claim 53 wherein the polysaccharide is selected from the group consisting of carboxymethylcellulose, carboxymethy ethyl cellulose, hyaluronic acid, carrageenan, alginic acid, sodium alginate, gellan gum, and mixtures thereof.

55. The composition for a wound dressing as set forth in claim 52 wherein the polyvalent cation is selected from a metal ion derived from salts selected from the group consisting of alkaline earth metal salts, alkali metal salts, transistion metal salts, and mixtures thereof.

56. The composition of claim 55 wherein the polyvalent cation is selected from the group consisting of calcium, barium, magnesium, zinc, iron, aluminum, copper, stronthium, manganese, and mixtures thereof.

57. The composition of claim 56, wherein the cation is selected from the group consisting of calcium, barium, copper, zinc, iron, aluminum, and mixtures thereof.

58. The composition of claim 57, wherein the cation is calcium.

59. The composition for a wound dressing as set forth in claim 52, wherein the upper limit of the DE of the calcium sensitive pectin is 50%.

60. The composition for a wound dressing as set forth in claim 52, wherein the upper limit of the DE of the calcium sensitive pectin is 30%.

61. The composition for a wound dressing as set forth in claim 52, wherein the lower limit of the DE of the calcium sensitive pectin is 0%.

62. The composition for a wound dressing as set forth in claim 52, wherein the lower limit of the DE of the calcium sensitive pectin is 5%.

63. The composition for a wound dressing as set forth in claim 52, wherein the lower limit of the DE of the calcium sensitive pectin is 10%.

64. The composition for a wound dressing as set forth in claim 52, wherein the upper limit of the degree of amidation (DA) of the calcium sensitive pectin is 40%.

65. The composition for a wound dressing as set forth in claim 52, wherein the upper limit of the DA of the calcium sensitive pectin is 25%.

66. The composition for a wound dressing as set forth in claim 52, wherein the upper limit of the DA of the calcium sensitive pectin is 20%.

67. The composition for a wound dressing as set forth in claim 52, wherein the lower limit of the DA of the calcium sensitive pectin is 0%.

68. The composition for a wound dressing as set forth in claim 52, wherein the lower limit of the DA of the calcium sensitive pectin is 5%.

69. The composition for a wound dressing as set forth in claim 52, wherein the lower limit of the DA of the calcium sensitive pectin is 10%.

70. The composition for a wound dressing as set forth in claim 52, wherein the calcium sensitive pectin is derived from citrus pectin.

71. The composition for a wound dressing prepared in accord with claim 32 in which a medicament is present in the polyvalent cation cross-linked pectin foam composition.

72. The composition for a wound dressing prepared in accord with claim 71 in which the medicament is collagen.

73. The composition for a wound dressing prepared in accord with claim 71 in which the medicament is maltodextrin.

74. The composition for a wound dressing prepared in accord with claim 71 in which the medicament is an antibiotic.

75. The composition for a wound dressing prepared in accord with claim 71 in which the medicament is an antibacterial agent.

76. The composition for a wound dressing prepared in accord with claim 71 in which the medicament is a hydrophilic substance.

77. The composition for a wound dressing prepared in accord with claim 76 in which the hydrophilic substance is a sodium acrylic polymer.
